(19) United States
(12) Patent Application Publication
(10) Pub. No.: US 2026/0047922 A1
(43) Pub. Date: Feb. 19, 2026

(54) CRIMPING ACCESSORY FOR VALVE LEAFLETS

(71) Applicant: MiRus LLC, Marietta, GA (US)

(72) Inventors: Jorge Jimenez, Marietta, GA (US); Elizabeth Smude, Marietta, GA (US)

(21) Appl. No.: 19/292,981

(22) Filed: Aug. 7, 2025

Related U.S. Application Data
(60) Provisional application No. 63/682,192, filed on Aug. 12, 2024.

(51) Int. Cl. A61F 2/24; A61F 2/95

(52) U.S. Cl. CPC A61F 2/2412; A61F 2/9524

(57) ABSTRACT

A device and method of inhibiting and preventing damage to leaflets during the crimping of a prosthetic valve, and/or facilitating the folding of the leaflets during the crimping of a prosthetic valve.

### REFERENCED APPLICATIONS

The present disclosure claims priority on U.S. Provisional Application Ser. No. 63/682,192 filed Aug. 12, 2024, which is incorporated herein by reference.

### FIELD OF DISCLOSURE

The present disclosure relates generally to an apparatus and method for crimping a medical device, particularly to an apparatus and method for crimping a prosthetic valve, more particularly to an apparatus and method that facilitate in reducing the incidence of damage to the leaflets of a prosthetic valve during the crimping of the frame of the prosthetic valve, and even more particularly to an apparatus and method that facilitate in reducing the incidence of damage to the leaflets of a prosthetic valve during the crimping of the frame of the prosthetic valve, while also facilitating in the reduction in the number and volume of void spaces between and about the leaflets and to reduce the crimped outer diameter profile of the prosthetic heat valve during the crimping process.

### BACKGROUND OF DISCLOSURE

Many cardiovascular devices such as stents, expandable heart valves and the like are inserted into a patient via the vascular system of a patient and then expanded at the treatment site. These devices are typically crimped onto catheter prior to insertion into a patient. The minimum diameter to which the cardiovascular device can be crimped onto the catheter will set a limit to the size of the cardiovascular passageway (e.g., blood vessel) to which the cardiovascular device can be inserted. Smaller crimp diameters can result in reduced damage to a blood vessel and/or organ (e.g., heart, etc.) when inserting into or to and/or placing the cardiovascular device at the treatment site. Smaller crimp diameters can also allow the cardiovascular device to be placed in smaller diameter blood vessels (e.g., blood vessels located in the brain, etc.).

The crimp diameter of the expandable cardiovascular device can be reduced by reducing the thickness and/or size of the frame, struts, etc. of the cardiovascular device. However, such reduction in size also affects the strength of the cardiovascular device after being expanded. After the cardiovascular device is expanded, it must retain its expanded shape at the treatment area, otherwise the cardiovascular device could become dislodged from the treatment area, could damage the treatment area, and/or fail to properly function at the treatment area. As such, cardiovascular devices formed of tradition materials such as stainless steel (e.g., 316L: 17-19 wt. % chromium, 13-15 wt. % nickel, 2-4 wt. % molybdenum, 2 wt. % max manganese, 0.75 wt. % max silicon, 0.03 wt. % max carbon, balance iron) and cobalt-chromium alloys (e.g., MP35N: 19-21 wt. % chromium, 34-36 wt. % nickel, 9-11 wt. % molybdenum, 1 wt. % max iron, 1 wt. % max titanium, 0.15 wt. % max manganese, 0.15 wt. % max silicon, 0.025 wt. % max carbon, balance cobalt) are required to maintain a frame and/or strut size/thickness that limits how small of crimping diameter can be obtained by the crimped cardiovascular device. Other types of CoCr alloys that have been used are Phynox and Elgiloy alloy (38-42 wt. % cobalt, 18-22 wt. % chromium, 14-18 wt. % iron, 13-17 wt. % nickel, 6-8 wt. % molybdenum), and L605 alloy (18-22 wt. % chromium, 14-16 wt. % W, 9-11 wt. % nickel, balance cobalt).

Medical devices such as Transcatheter aortic valves (TAVs) represent a significant advancement in prosthetic heart valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; and US Pub. No. 2020/0405482, all of which are incorporated fully herein by reference.

A TAV is designed to be compressed into a small diameter catheter, remotely placed within a patient's diseased aortic valve to take over the function of the native valve. Some TAVs are balloon-expandable, while others are self-expandable. In both cases, the TAVs are deployed within a calcified native valve that is forced permanently open and becomes the surface against which the frame is held in place by friction. TAVs can also be used to replace failing bioprosthetic or transcatheter valves, commonly known as a valve in valve procedure. Major TAVR advantages to the traditional surgical approaches include refraining from cardiopulmonary bypass, aortic cross-clamping and sternotomy which significantly reduces patients' morbidity.

TAVR involves delivery, deployment, and implantation of a crimped, framed valve within a diseased aortic valve or degenerated bioprosthesis. Some limitation of the current procedure for TAVR include damage to the leaflets and/or frame during crimping of the frame of the prosthetic heart valve, and b) device failure. TAVR involves delivery, deployment, and implantation of a crimped valve frame within a diseased aortic valve or degenerated bioprosthesis. One limitation of these types of procedures is the diameter to which the valve frame can be crimped without damaging the leaflet tissues within, and vascular complications such as dissection due to the size of the delivery system.

As such, there has been an ongoing need for an improved medical device that can minimize damage to the leaflets during the crimping process.

### SUMMARY OF THE DISCLOSURE

The present disclosure relates to a device for use with crimper systems and methods for crimping a medical such as, but not limited to, a prosthetic valve (e.g., prosthetic heart valve, etc.), and more particularly to devices and methods that facilitate in the desired folding of the leaflets of a prosthetic valve during the crimping of the frame of the prosthetic valve to reduce the risk of damage to the leaflets during the crimping process.

In another one non-limiting aspect of the present disclosure, there is provided a crimping accessory that can be used with a crimper device to facilitate in obtaining a leaflet folding configuration or arrangement during the crimping of the frame of a prosthetic valve so as to obtain a desired folded leaflet configuration after the frame has been fully crimped and/or to reduce or prevent damage to the leaflets during the crimping process. In one non-limiting embodiment, the crimping accessory is configured to inwardly bend one or more or all of the ends and/or end portions of the leaflets that are located at or near the outflow end of the prosthetic valve toward the central axis of the frame of the prosthetic valve prior to and/or during the crimping of the frame of the prosthetic valve. Such bending of the one or more leaflets by the crimping accessory occurs a) prior to the initial crimping of the frame of the prosthetic valve, and/or b) during the partial or full crimping of the frame of the prosthetic valve. One or more portions of the crimping accessory can optionally include a coating. Such coating when used, can be used to a) limit or prevent damage to the frame and/or leaflets of the prosthetic valve when one or more portions of the crimping accessory engages the frame and/or leaflets of the prosthetic valve during the crimping process, b) limit or prevent contamination of the frame and/or leaflets of the prosthetic valve when one or more portions of the crimping accessory engages the frame and/or leaflets of the prosthetic valve during the crimping process, and/or c) reduce friction between one or more portions of the crimping accessory and the frame and/or leaflets of the prosthetic valve during the crimping process. The type and thickness of the coating, when used, is non-limiting. In one non-limiting embodiment, the coating is formed of a polymer material (e.g., PTFE [Polytetrafluoroethylene], polyethylene, polyvinylidene fluoride, etc.). In another non-limiting embodiment, the thickness of the coating, when used, is at least 0.001 microns (e.g., 0.001-500 microns and all values and ranges therebetween).

In another one non-limiting aspect of the present disclosure, there is provided a crimping accessory that can be used with a crimper device to a) facilitate in obtaining a leaflet folding configuration or arrangement during the crimping of the frame of a prosthetic valve so as to obtain a desired folded leaflet configuration after the frame has been fully crimped and/or b) reduce or prevent damage to the leaflets during the crimping process. The crimping accessory can be configured to be removed from or disengaged from the one or more leaflets prior to the outflow end of the frame of the prosthetic valve being 5-99% fully crimped (and all values and ranges therebetween).

In accordance with another non-limiting aspect of the present disclosure, there is provided a crimping accessory and method for folding the leaflets on a prosthetic valve that includes the step of inserting the crimping accessory at least partially in the interior of the prosthetic valve prior to the crimping of the prosthetic valve and/or after the partial crimping of the prosthetic valve. In one non-limiting embodiment, the crimping accessory includes two or more wires that are configured to engage a portion of two or more leaflets as the prosthetic valve is partially or fully crimped. The wires of the crimping accessory can be configured such that one or more or all of the wires can be inserted 10-100% (and all values and ranges therebetween) along the longitudinal length of the prosthetic valve. The wires can be configured such that two or more or all of the wires have the same shape, size, length, width, cross-sectional shape, and/or configuration. The wires can be oriented such that two or more of the adjacently positioned wires have the same spacing from one another. In one non-limiting arrangement, the width and/or thickness of one or more or all of the wires is 0.01-8 mm (and all values and ranges therebetween; 0.05-5 mm, etc.). The one or more wires are generally configured to be flexible so the one or more wires can bend as the prosthetic valve is partially or fully crimped. The material used to form the crimping accessory is non-limiting (e.g., metal, plastic, paper, composite material, etc.). The end region of one or more of the wires can optionally include a foam and/or plastic region to protect the leaflets from damage of the wires. The shape and size of the foam and/or plastic regions is non-limiting. The color of the foam and/or plastic region is non-limiting (e.g., clear, white, etc.). The foam and/or plastic region and/or the wires can optionally include one or more markers (e.g., colored region, physical shaped feature, etc. that can be used to facilitate orienting the foam and/or plastic region and/or the wires relative to one or more leaflets. In another non-limiting configuration, the foam and/or plastic region is formed of a low friction material (e.g., coefficient of friction (COF) of 0.01 to 0.6 and all values and ranges therebetween).

In accordance with another non-limiting aspect of the present disclosure, during use, the wires (which may or may not include a coating material) or the crimping accessory are inserted into the interior of the prosthetic valve along a portion or the complete longitudinal length of the prosthetic valve prior to crimping the prosthetic valve. Generally, at least a portion of the wires (coat or uncoated) are positioned between a portion of the leaflet and the frame so as to facilitate spacing at least a portion of the leaflet from the frame during a portion or all of the crimping process of the frame. As the prosthetic valve is partially crimped, the wires on the crimping accessory engage one or more leaflets and a) cause the leaflets to initially fold in a certain way based on the engagement of the leaflets with the wires of the crimping accessory as the frame is crimped, and/or b) cause at least a portion of the leaflets to be spaced from the frame so as to inhibit or prevent damage to the one or more leaflets during the crimping process (e.g., preventing a portion of the leaflets from moving into one or more side openings of the frame and becoming pinched and/or torn as the frame is crimped, etc.). In one non-limiting embodiment, during the partial crimping of the prosthetic valve, the wires of the crimping accessory can be used to optionally cause one or more of the leaflets to partially bend due to the inwardly radial forces applied by the wires on the leaflets. Once the prosthetic valve is partially crimped, the crimping accessory can optionally be partially or fully removed from the interior of the prosthetic valve, and thereafter the prosthetic valve can be subjected to a final crimping process to fully crimp the prosthetic valve. During the final crimping process, the leaflets are a) continued to be caused to fold in a desired manner to reduce or minimize the volume of the leaflets after the prosthetic valve is fully crimped, and/or b) little or no damage to the leaflets occurs since after the frame has been partially crimped, the openings in the sides of the frame have been partially crimped and generally reduced in size, thus the openings are too small for a portion of the leaflet to enter the reduced sized openings and be pinched, torn or otherwise damaged as the frame is continued to be crimped to the final crimped state. During the crimping process that involves the use of the crimping accessory, the type of crimping process used to crimp the prosthetic valve while the radially collapsible insert is partially or fully inserted in the prosthetic valve is non-limiting. Such crimping processes can include a) a traditional prior art crimping process wherein the complete prosthetic valve is subjected to crimping forces, b) a stepwise crimping process wherein one portion of the prosthetic valve is subjected to crimping forces and then subsequently other portions of the prosthetic valve are subjected to crimping forces, and/or c) a progressive continuous crimping process wherein the crimping of the prosthetic valve starts at the inflow end or the outflow end and the crimping continuously progresses along the longitudinal length of the prosthetic valve to the opposite end of the prosthetic valve. After the crimping accessory is removed from the prosthetic valve, the crimping processes that can be used to complete the crimping of the prosthetic valve can include a) a traditional prior art crimping process wherein the complete prosthetic valve is subjected to crimping forces, b) a stepwise crimping process wherein one portion of the prosthetic valve is subjected to crimping forces and then subsequently other portions of the prosthetic valve are subjected to crimping forces, and/or c) a progressive continuous crimping process wherein the crimping of the prosthetic valve starts at the inflow end or the outflow end and the crimping continuously progresses along the longitudinal length of the prosthetic valve to the opposite end of the prosthetic valve. In one non-limiting method, during use of the crimping accessory while it is at least partially inserted in the prosthetic valve, the crimping accessory can be held or mounted to not rotate during the partial crimping of the prosthetic valve. In another non-limiting method, during use of the crimping accessory while it is at least partially inserted in the prosthetic valve, the crimping accessory can be allowed to rotate during the partial crimping of the prosthetic valve. In non-limiting embodiment, the crimping accessory includes at least two wires. Each of the two or more wires (e.g., 2 wires, 3 wires, 4 wires, etc.) have generally the same shape, thickness, length, width, and configuration. If one or more of the wires has a coating, foam and/or plastic region, the thickness, shape and/or size of the coating, foam and/or plastic region on each of two or more wires can be the same or different. In one non-limiting configuration, the crimping accessory includes 3 wires, and wherein the three wires are spaced such that adjacently positioned wires are equally spaced from one another. Each of the wires is configured to extend 10-100% (and all values and ranges therebetween) the longitudinal length of the interior of the prosthetic valve. Each of the wires has a thickness and/or width of 0.02-3 mm (and all values and ranges therebetween).

In another non-limiting aspect of the present disclosure, there is provided a crimping accessory that can optionally be used with the crimper device in accordance with the present disclosure to facilitate in a) obtaining a leaflet folding configuration or arrangement during the crimping of the frame of a prosthetic valve and/or b) inhibit or prevent damage to the leaflets during the crimping of the frame of the prosthetic valve, and wherein the crimping accessory includes a handle portion and one or more leaflet engagement members that are attached to and extend from the handle portion. In one non-limiting configuration, the handle portion is sized and shaped such that it can be grasped by a user to enable the user to position the one or more leaflet engagement members into a portion of the prosthetic valve. In another non-limiting configuration, the one or more leaflet engagement members are attached to the distal end or distal end portion of the handle portion. The type of connection used to attach one or more leaflet engagement members to the handle is non-limiting. In one non-limiting configuration, the one or more leaflet engagement members extend radially outwardly (e.g., 5-45° radially outwardly and all values and ranges therebetween) from the central longitudinal axis of the handle portion. When the crimping accessory includes two or more leaflet engagement members, two or more or all of the leaflet engagement members can a) extend radially outwardly from the central longitudinal axis of the handle portion at within ±5° of the same angle from one another, and/or b) have the same size, shape, and/or be formed of the same material; however, this is not required. In another non-limiting configuration, one or more of the leaflet engagement members are formed of a flexible material that enables the one or more leaflet engagement members to flex and/or bend a) when positioning the one or more leaflet engagement members about one or more leaflets, and/or b) during the crimping of the frame of the prosthetic valve and while the one or more leaflet engagement members are still engaged with the one or more leaflets during the crimping of the frame. In another non-limiting configuration, the one or more leaflet engagement members are each formed of a wire or wire loop (e.g., metal wire loop, plastic wire loop, etc.). In another non-limiting embodiment, the crimping accessory is moved along the longitudinal axis of the frame and toward the frame until the one or more leaflet engagement members engage the end or end portion of the one or more leaflets. Thereafter, the crimping accessory continues to be moved along the longitudinal axis of the frame such that the end or end portion of the one or more leaflet engagement members move between a portion or all of the one or more leaflets and the inner surface of the frame. In one non-limiting method, as the crimping accessory continues to be moved along the longitudinal axis of the frame, the angular orientation of the one or more leaflet engagement members relative to the central axis of the handle portion of the crimping accessory causes the end and/or end portions of the leaflets to be bent toward the central axis of the frame. Generally, the size and configuration of the end region of the one or more leaflet engagement members inhibits or prevents the end or end region of the one or more leaflet engagement members from passing through the side openings in the frame of the prosthetic valve. Furthermore, the one or more leaflet engagement members generally are inserted only through a portion of the longitudinal length of the frame (e.g., 1-80% of the longitudinal length and all values and ranges therebetween) prior to and/or during the crimping process of the frame. In one non-limiting method of use, one or more leaflet engagement members are inserted only through a portion of the longitudinal length of the frame and are spaced from the region of the frame wherein the one or more leaflets are connected to the frame during the crimping of the frame.

In another non-limiting aspect of the present disclosure, the medical device is a valve (e.g., heart valve, TAVR valve, aortic, mitral valve replacement, tricuspid valve replacement, pulmonary valve replacement, etc.). In one non-limiting embodiment, the medical device includes an expandable frame, more particularly the medical device is in the form of a cardiovascular implant for the treatment of structural heart disease wherein the cardiovascular implant includes an expandable frame, and still more particularly to a medical device is in the form of a prosthetic valve for the for the treatment of structural heart disease wherein the prosthetic valve includes an expandable frame that is optionally formed of a rhenium containing metal alloy.

In another non-limiting aspect of the present disclosure, the medical device is a valve that includes a frame that is formed of a rhenium containing metal alloy. The frame of the prosthetic valve is optionally partially (e.g., 10-99.99 wt. % and all values and ranges therebetween) or fully formed of the rhenium containing alloy. In one non-limiting aspect of the disclosure, the prosthetic valve (e.g., heart valve, TAVR valve, mitral valve replacement, tricuspid valve replacement, pulmonary valve replacement, etc.) includes a radially collapsible and expandable frame and a leaflet structure that comprises a plurality of leaflets. In another non-limiting embodiment, the prosthetic valve optionally includes an annular skirt or cover member that is disposed on and partially or fully covering or overlaid over the cells of at least a portion of the frame.

In another and/or alternative non-limiting aspect of the disclosure, the frame of the prosthetic valve is optionally partially or fully formed of a) a refractory metal alloy and/or b) a metal alloy that includes at least 15 atomic weight percent (awt. %) or atomic percent (awt. %) rhenium so as to create a “rhenium effect” in the metal alloy. As used herein, atomic weight percent (awt. %) or atomic percentage (awt. %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of at least 15 awt. % rhenium results in the ductility and/or tensile strength of the metal alloy to improve as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc. In one non-limiting arrangement, 50-100 wt. % (and all values and ranges therebetween) of the expandable frame of the prosthetic valve is formed of a refractory metal alloy or a metal alloy that includes at least 15 awt. % rhenium. In another non-limiting arrangement, the metal alloy that is used to partially or fully form the expandable frame of the prosthetic valve includes at least 30 wt. % (e.g., 30-99 wt. % and all values and ranges therebetween) of one or more of molybdenum, rhenium, niobium, tantalum or tungsten. In another and/or alternative non-limiting embodiment, the frame of the prosthetic valve is optionally partially (e.g. 1-99.999 wt. % and all values and ranges therebetween) or fully formed of a metal material that includes a) stainless steel, b) CoCr alloy, c) TiAlV alloy, d) aluminum alloy, c) nickel alloy, f) titanium alloy, g) tungsten alloy, h) molybdenum alloy, i) copper alloy, j) beryllium-copper alloy, k) titanium-nickel alloy, 1) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 atomic weight percent (awt. %) or atomic percent (awt. %) rhenium (e.g., 5-99 awt. % rhenium and all values and ranges therebetween). In accordance with another and/or alternative non-limiting embodiment, the metal alloy that is used to partially or fully form the frame of the prosthetic valve includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium effect.

In accordance with another and/or alternative non-limiting aspect of the present disclosure, there is provided a medical device that includes a frame at least partially formed of a plurality of angularly spaced, vertically extending posts, or struts. The posts or struts can optionally be interconnected via a lower row of circumferentially extending struts and an upper row of circumferentially extending struts. The struts can be arranged in a variety of patterns (e.g., zig-zag pattern, saw-tooth pattern, triangular pattern, polygonal pattern, oval pattern, etc.). One or more of the posts and/or struts can have the same or different thicknesses and/or cross-sectional shape and/or cross-sectional area.

In accordance with another and/or alternative non-limiting aspect of the present disclosure, there is provided a medical device that includes a frame that can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the struts on the frame and/or to fill-in the openings between the struts on the frame.

In accordance with another and/or alternative non-limiting aspect of the present disclosure, there is provided a medical device in the form of a prosthetic valve that includes an inner skirt that can be formed of a variety of flexible materials (e.g., polymer [e.g., polyethylene terephthalate (PET), polyester, nylon, Kevlar,®, silicon, etc.], composite material, metal, fabric material, etc.). In one non-limiting embodiment, the material used to partially or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with a flexible material or with a stretchable and/or compressible material so as to provide additional structural integrity to the inner skirt. The size, configuration, and thickness of the inner skirt is non-limiting (e.g., thickness of 0.1-20 mils and all values and ranges therebetween). The inner skirt can be secured to the inside and/or outside of the frame using various means (e.g., sutures, clamp arrangement, etc.).

In accordance with another and/or alternative non-limiting aspect of the present disclosure, there is provided a medical device in the formed of a prosthetic valve that optionally includes an inner skirt that can be used to 1) at least partially seal and/or prevent perivalvular leakage, 2) at least partially secure the leaflet structure to the frame, 3) at least partially protect the leaflets from damage during the crimping and/or expansion process, and/or 4) at least partially protect the leaflets from damage during the operation of the prosthetic valve in the heart.

In accordance with another and/or alternative non-limiting aspect of the present disclosure, there is provided a medical device in the form of a prosthetic valve that optionally includes an outer or sleeve that is positioned at least partially about the exterior region of the frame. The outer skirt or sleeve generally is positioned completely around a portion of the outside of the frame. Generally, the outer skirt is positioned about the lower portion of the frame, but does not fully cover the upper half of the frame; however, this is not required. The outer skirt can be connected to the frame by a variety of arrangements (e.g., sutures, adhesive, melted connection, clamping arrangement, etc.). At least a portion of the outer skirt can optionally be located on the interior surface of the frame. Generally, the outer skirt is formed of a more flexible and/or compressible material than the inner skirt; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material to provide additional structural integrity to the outer skirt. The size, configuration, and thickness of the outer skirt is non-limiting. The thickness of the outer skirt is generally 0.1-20 mils (and all values and ranges therebetween).

In accordance with another and/or alternative non-limiting aspect of the present disclosure, there is provided a medical device in the form of a prosthetic valve that includes a leaflet structure that can be attached to the frame and/or skirt. The connection arrangement used to secure the leaflet structures to the frame and/or skirt is non-limiting (e.g., sutures, melted bold, adhesive, clamp arrangement, etc.). The material used to form the leaflet structures include bovine pericardial tissue, biocompatible synthetic materials, or various other suitable natural or synthetic materials.

In accordance with another and/or alternative non-limiting aspect of the present disclosure, there is provided a medical device in the form of a prosthetic valve that includes a leaflet structure comprised of two or more leaflets (e.g., 2, 3, 4, 5, 6, etc.). In one non-limiting arrangement, the leaflet structure includes three leaflets arranged to collapse in a tricuspid arrangement. The configuration of the leaflet structures is non-limiting.

In accordance with another and/or alternative non-limiting aspect of the present disclosure, there is provided a medical device in the form of a prosthetic valve that includes a leaflet structure wherein the leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, etc.).

In accordance with another and/or alternative non-limiting aspect of the present disclosure, there is provided a medical device in the form of a prosthetic valve that includes a leaflet structure wherein one or more of the leaflets can optionally include reinforcing structures or strips to 1) facilitate in securing the leaflets together, 2) facilitate in securing the leaflets to the skirt and/or frame, and/or 3) inhibit or prevent tearing or other types of damage to the leaflets.

In accordance with another and/or alternative non-limiting aspect of the present disclosure, the medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. The type and/or amount of agent included in medical device and/or coated on medical device can vary. When two or more agents are included in and/or coated on medical device, the amount of two or more agents can be the same or different. The type and/or amount of agent included on, in and/or in conjunction with medical device are generally selected to address one or more clinical events.

In accordance with another and/or alternative non-limiting aspect of the present disclosure, the one or more agents on and/or in the medical device, when used on the medical device, can be released in a controlled manner so the area in question to be treated is provided with the desired dosage of agent over a sustained period of time. Non-limiting arrangements that can be used to control the release of one or more agents from the medical device include 1) at least partially coat one or more agents with one or more polymers, 2) at least partially incorporate and/or at least partially encapsulate one or more agents into and/or with one or more polymers, and/or 3) insert one or more agents in pores, passageway, cavities, etc. in the medical device and at least partially coat or cover such pores, passageway, cavities, etc. with one or more polymers. As can be appreciated, other or additional arrangements can be used to control the release of one or more agents from the medical device.

In accordance with another and/or alternative non-limiting aspect of the present disclosure, the medical device can optionally include a marker material that facilitates enabling the medical device to be properly positioned in a body passageway (e.g., blood vessel, heart valve, etc.). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.).

In accordance with another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, laser micro-machining, micro-molding, 3D printing, etc.); however, other or additional manufacturing techniques can be used.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology (e.g., 3D printing, etc.). In accordance with another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie-like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm. As can be appreciated, when the medical device includes one or more surface structures, 1) all the surface structures can be micro-structures, 2) all the surface structures can be non-micro-structures, or 3) a portion of the surface structures can be micro-structures and a portion can be non-micro-structures.

In accordance with another and/or alternative non-limiting aspect of the present disclosure, the medical device can optionally be an expandable device that can be expanded by use of some other device (e.g., balloon, etc.).

In accordance with another and/or alternative non-limiting aspect of the present disclosure, the medical device can optionally be fabricated from a material having no or substantially no shape-memory characteristics.

In accordance with another and/or alternative non-limiting aspect of the present disclosure, there is provided a prosthetic valve that is configured to be inserted into a desired location in the body (e.g., the aortic valve, tricuspid valve, pulmonary valve, mitral valve). The frame of the prosthetic valve can be at least partially formed of a plastically-expandable material that permits crimping of the frame to a smaller profile for delivery and expansion of the prosthetic valve to a larger profile. The expansion of the crimped frame can optionally be an expansion device such as, but not limited to, a balloon of on a balloon catheter. As can be appreciated, the medical device can be a device other than a prosthetic valve (e.g., stent, etc.) that includes a frame that is at least partially formed of a plastically expandable material that permits crimping of the frame to a smaller profile for delivery and expansion of the medical device to a larger profile.

One non-limiting object of the present disclosure is the provision of a device and method for facilitating in the crimping of a crimpable or plastically deformable portion of a medical device that includes one or more leaflets.

Another and/or alternative non-limiting object of the present disclosure is the provision of a device and method for crimping a crimpable or plastically deformable portion of a medical device that includes one or more leaflets so as to a) reduce void spaces about the leaflets after completion of the crimping of the crimpable or plastically deformable portion of a medical device, and/or b) reduce or prevent damage to the leaflets of the medical device during the crimping of the medical device.

Another and/or alternative non-limiting object of the present disclosure is the provision of a device and method for crimping a crimpable or plastically deformable portion of a medical device that includes one or more leaflets so as to a) obtain a desired folding profile of the leaflets after completion of the crimping of the crimpable or plastically deformable portion of a medical device, and/or b) reduce or prevent damage to the leaflets of the medical device during the crimping of the medical device.

Another and/or alternative non-limiting object of the present disclosure is the provision of a method for reducing the profile of a frame of a medical device wherein the medical device is a prosthetic valve; the crimpable or plastically deformable portion of the medical device includes a frame of the prosthetic valve; the one or more leaflets are connected to the frame.

Another and/or alternative non-limiting object of the present disclosure is the provision of a method for reducing the profile of a frame of a medical device further including the step of causing at least a portion of one or more of the leaflets to be bent toward a central longitudinal axis of the crimpable or plastically deformable portion of the medical device a) prior to applying the crimping force on the crimpable or plastically deformable portion of the medical device, and/or b) while applying the crimping force to the crimping of the crimpable or plastically deformable portion of the medical device.

Another and/or alternative non-limiting object of the present disclosure is the provision of a method for reducing the profile of a frame of a medical device further includes the step of applying a rotational force about a central longitudinal axis of the crimpable or plastically deformable portion to one or more of the leaflets a) prior to applying the crimping force on the crimpable or plastically deformable portion of the medical device, and/or b) while applying the crimping force to the crimping of the crimpable or plastically deformable portion of the medical device.

Another and/or alternative non-limiting object of the present disclosure is the provision of a method for reducing the profile of a frame of a medical device further providing a crimping accessory that is configured to a) facilitate in folding the one or more of the leaflets during the step of applying the crimping force on the crimpable or plastically deformable portion of the medical device, and/or b) reduce or prevent damage to the leaflets of the medical device during the crimping of the medical device.

Another and/or alternative non-limiting object of the present disclosure is the provision of a method for reducing the profile of a frame of a medical device further providing a crimping accessory that is configured to a) facilitate in folding the one or more of the leaflets and/or b) reduce or prevent damage to the leaflets of the medical device during the step of applying the crimping force on the crimpable or plastically deformable portion of the medical device, wherein the crimping accessory is positioned within the leaflets such that the leaflets are positioned between the frame of the prosthetic valve and the crimping accessory.

Another and/or alternative non-limiting object of the present disclosure is the provision of a method for reducing the profile of a frame of a medical device further including the step of positioning at least a portion of the crimping accessory between the one or more leaflets and the crimpable or plastically deformable portion of the medical device to facilitate in a) bending at least a portion of one or more of the leaflets toward the central longitudinal axis of the crimpable or plastically deformable portion, and/or b) rotating at least a portion of one or more of the leaflets about the central longitudinal axis of the crimpable or plastically deformable portion.

Another and/or alternative non-limiting object of the present disclosure is the provision of a method for reducing the profile of a frame of a medical device further includes the step of fully disengaging the crimping accessory from one or more of the leaflets prior to completion of applying the crimping force on the crimpable or plastically deformable portion of the medical device.

Another and/or alternative non-limiting object of the present disclosure is the provision of a method for reducing the profile of a frame of a medical device further including the step of positioning a portion or one or more of the leaflet engagement members between a portion of one or more of the leaflets and the crimpable or plastically deformable portion of the medical device to cause at least a portion of the one or more of the leaflets to bend towards the central longitudinal axis of the crimpable or plastically deformable portion of the medical device.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for case of recognition in the drawings. Reference may now be made to the drawings, which illustrate various embodiments that the disclosure may take in physical form and in certain parts and arrangement of parts wherein:

FIG. 1 is an illustration of a TAV.

FIG. 2 is an illustration of a TAV that includes an inner skirt and leaflet structure.

FIGS. 3-4 illustrate a non-limiting crimper device that can be used to crimp a prosthetic valve;

FIG. 5 illustrates the folding profile of leaflets in a partially crimped prosthetic valve by use of the crimping process in accordance with the present disclosure wherein the leaflets are being to align so that void spaces are reduced once the frame is fully crimped;

FIG. 6 illustrates the folding profile of leaflets in the fully crimped prosthetic valve, wherein the prosthetic valve was crimped by the crimper device in accordance with the present disclosure and wherein the leaflets were caused to be folded in accordance with the one or more methods of the present disclosure, and wherein there is a reduction of void spaces about the folded leaflets when the frame valve is in the fully crimped state;

FIGS. 7A-7C illustrates one non-limiting crimping method for progressively or stepwise crimping a prosthetic valve to obtain a leaflet folding configuration or arrangement by use of the crimper device in accordance with the present disclosure so as to obtain a leaflet folding configuration or arrangement of FIGS. 5-6;

FIG. 8 illustrates a non-limiting crimping accessory that can optionally be used with the crimper device in accordance with the present disclosure to facilitate in obtaining a leaflet folding configuration or arrangement during the crimping of the frame of a prosthetic valve so as to obtain a folded leaflet configuration after the frame has been fully crimped as illustrated in FIGS. 5-6;

FIG. 9 illustrates the outflow end of a fully crimped frame of a prosthetic valve and wherein the arrows illustrate the counter-clockwise folding profile pattern of the leaflets so as to reduce the void spaced about the leaflets, and wherein the counter-clockwise folding profile pattern of the leaflets by the use of the crimping method in accordance with the present disclosure and the optional use of the crimping accessory illustrated in FIGS. 10-12;

FIGS. 10-12 illustrates another crimping accessory that includes a plurality of wires that can be used in a crimping method for crimping a prosthetic valve to obtain a leaflet folding configuration or arrangement that is the same or similar to the leaflet folding configuration or arrangement of FIGS. 5-6.

### DESCRIPTION OF NON-LIMITING EMBODIMENTS

A more complete understanding of the articles/devices, processes and components disclosed herein can be obtained by reference to the accompanying drawings. These figures are merely schematic representations based on convenience and the case of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

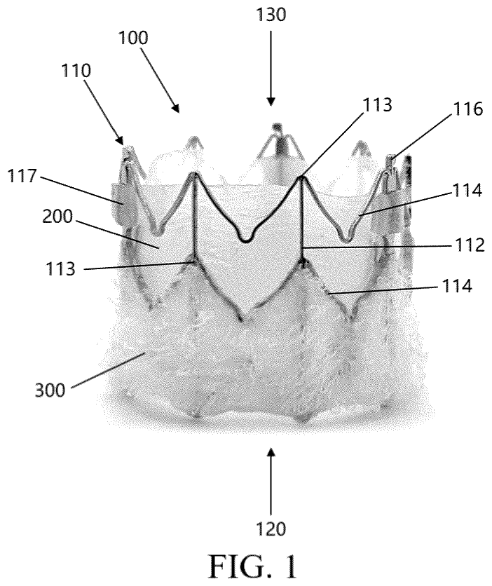

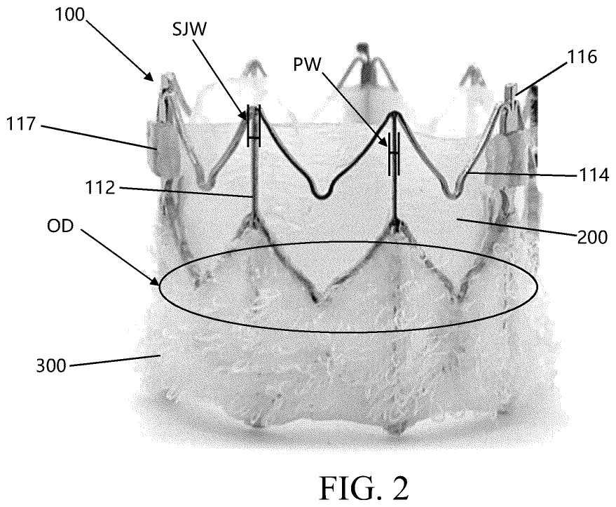

Referring now to FIGS. 1-2, these figures are illustrations of an implantable prosthetic valve 100 (e.g., TAV) and a method for inserting the prosthetic valve 100 in a valve region A (e.g., aortic valve, etc.) of a heart. Prosthetic valve 100 can be implanted in the annulus of native aortic valve A; however, prosthetic valve 100 also can be configured to be implanted in other valves of the heart. Although the medical device illustrated is a TAV, the present disclosure is not limited to TAVs or any other heart valve replacement.

The prosthetic valve 100 generally comprises a frame 110 formed of a plurality of axial longitudinal members and angular articulating members 112, 114 strut joints 113, leaflet structure 200 supported by frame 110, and an inner skirt 300 secured to the outer surface of frame 110 and/or leaflet structure 200. The frame can include one or more an orientation structures or commissural markers 116. The frame 110 is partially or fully formed of a rhenium containing metal alloy. Prosthetic valve 100 has a “lower” end 120 and an “upper” end 130, wherein lower end 120 of prosthetic valve 100 is the inflow end and the upper end 130 of prosthetic valve 100 is the outflow end.

Frame 110 can optionally be coated with a polymer material (e.g., silicone, PTFE, ePTFE, polyurethane, polyolefins, hydrogels, biological materials [e.g., pericardium or biological polymers such as collagen, gelatin, or hyaluronic acid derivatives], etc.). The coating can be used to partially or fully encapsulate one or more of the vertically extending axial longitudinal members 112 and/or non-vertically angular articulating members 114 on frame 110 and/or to partially or fully fill-in one or more of the openings between the non-vertically angular articulating members 114 and/or vertically extending axial longitudinal members 112.

The inner skirt 300 can be formed of a variety of flexible materials (e.g., polymer (e.g., polyethylene terephthalate (PET), polyester, nylon, Kevlar, silicon, etc.), composite material, metal, fabric material, etc. In one non-limiting embodiment, the material used to partially or fully form inner skirt 300 can be substantially non-clastic (i.e., substantially non-stretchable and non-compressible). In another non-limiting embodiment, the material used to partially or fully form inner skirt 300 can be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). Inner skirt 300 can optionally be formed from a combination of a cloth or fabric material that is coated with a flexible material or with a stretchable and/or compressible material so as to provide additional structural integrity to inner skirt 300. The size, configuration, and thickness of inner skirt 300 is non-limiting (e.g., thickness of 0.1-20 mils and all values and ranges therebetween). The inner skirt 300 can be secured to the inside and/or outside of the frame 110 using various means (e.g., sutures, clips, clamp arrangement, etc.).

Inner skirt 300 can be used to 1) at least partially seal and/or prevent perivalvular leakage, 2) at least partially secure leaflet structure 200 to frame 110, 3) at least partially protect one or more of the leaflets of leaflet structure 200 from damage during the crimping process of prosthetic valve 100, 4) at least partially protect one or more of the leaflets of leaflet structure 200 form damage during the operation of prosthetic valve 100 in a heart.

Prosthetic valve 100 can optionally include an outer skirt or sleeve (not shown) that is positioned at least partially about the exterior region of frame 110. The outer skirt or sleeve (when used) generally is positioned completely around a portion of the outside of frame 110. Generally, the outer skirt is positioned about the lower portion of frame 110 and does not fully cover the upper portion of frame 110; however, this is not required. The outer skirt can be connected to frame 110 by a variety of arrangements (e.g., sutures, adhesive, melted connection, clamping arrangement, etc.). At least a portion of the outer skirt can optionally be located on the interior surface of frame 110; however, this is not required. Generally, the outer skirt is formed of a more flexible and/or compressible material than inner skirt 300; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material so as to provide additional structural integrity to the outer skirt. The size, configuration, and thickness of the outer skirt is non-limiting. The thickness of the outer skirt is generally 0.1-20 mils (and all values and ranges therebetween).

Leaflet structure 200 can be attached to frame 110 and/or inner skirt 300. The connection arrangement used to secure leaflet structure 200 to frame 110 and/or inner skirt 300 is non-limiting (e.g., sutures, melted bold, adhesive, clamp arrangement, etc.). The material used to form the one or more leaflets of leaflet structure 200 include, but are not limited to, bovine pericardial tissue, biocompatible synthetic materials, or various other suitable natural or synthetic materials.

Leaflet structure 200 can be comprised of two or more leaflets (e.g., 2, 3, 4, 5, 6, etc.). In one non-limiting arrangement, leaflet structure 200 includes three leaflets that are arranged to collapse in a tricuspid arrangement. The size, shape and configuration of the one or more leaflets of leaflet structure 200 are non-limiting. In one non-limiting arrangement, the leaflets have generally the same shape, size, configuration and thickness.

Two of more of the leaflets of leaflet structure 200 can optionally be secured to one another at their adjacent sides to form commissures of leaflet structure 200 (the edges where the leaflets come together). Leaflet structure 200 can be secured to frame 110 and/or inner skirt 300 by a variety of connection arrangement (e.g., sutures, adhesive, melted bond, clamping arrangement, etc.).

One or more leaflets of the leaflet structure 200 can optionally include reinforcing structures or strips to 1) facilitate in securing the leaflets together, 2) facilitate in securing the leaflets to the inner skirt 300 and/or frame 110, and/or 3) inhibit or prevent tearing or other types of damage to the leaflets.

Prosthetic valve 100 is configured to be radially collapsible to a collapsed or crimped state for introduction into the body on a delivery catheter and radially expandable to an expanded state for implanting prosthetic valve 100 at a desired location in the heart (e.g., aortic valve, etc.). The frame of prosthetic valve 100 is made of a plastically-expandable material (e.g., metal alloy) that permits crimping of the frame to a smaller profile for delivery and expansion of prosthetic valve 100 using an expansion device. The prosthetic valve can be crimped on a balloon catheter. The balloon on the balloon catheter can be used to expand the frame from a crimped state to an expanded state. Various types of crimping apparatus and techniques can be used to crimp the prosthetic valve on the balloon delivery catheter. The process of crimping a prosthetic valve using a crimping device is known in the art and will not be described herein.

Once prosthetic valve 100 is crimped on the balloon of the balloon catheter, the balloon catheter is inserted through a blood vessel and to the location in the heart wherein prosthetic valve 100 is to be deployed, and thereafter the balloon on the balloon catheter is expanded to thereby cause prosthetic valve 100 to be expanded and secured in a valve region of the heart. Thereafter, the balloon is deflated and the balloon catheter is removed from the patient.

Referring now to FIG. 2, the post width PW and/or the strut joint width SJW of a frame 110 that are formed of a refractory metal alloy or an alloy that includes at least 15 awt. % rhenium in accordance with the present disclosure can be smaller than the post width PW and/or the strut joint width SJW of a frame formed of stainless steel, nitinol, Co—Cr alloy or TiAlV alloy, and still have the same or greater radial strength when the frame is expanded as compared to a frame formed of stainless steel, nitinol, Co—Cr alloy or TiAlV alloy. A frame having smaller post widths and strut joint widths can be used in frames formed of that are formed of a refractory metal alloy or an alloy that includes at least 15 awt. % rhenium as compared to frames formed of stainless steel, CoCr, Nitinol, and TiAlV alloy without sacrificing the strength of the frame.

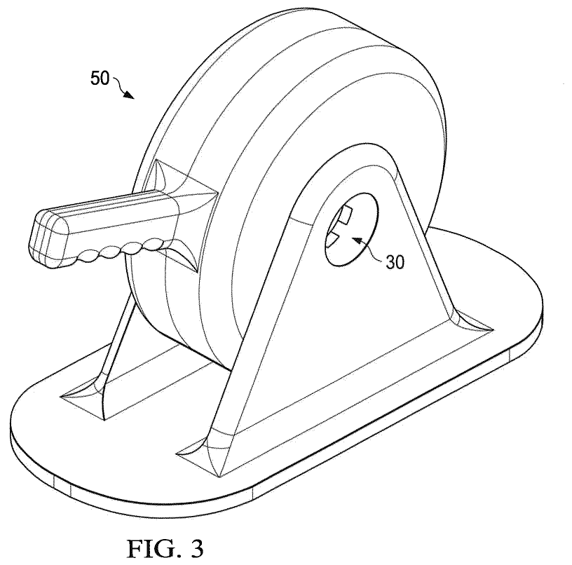

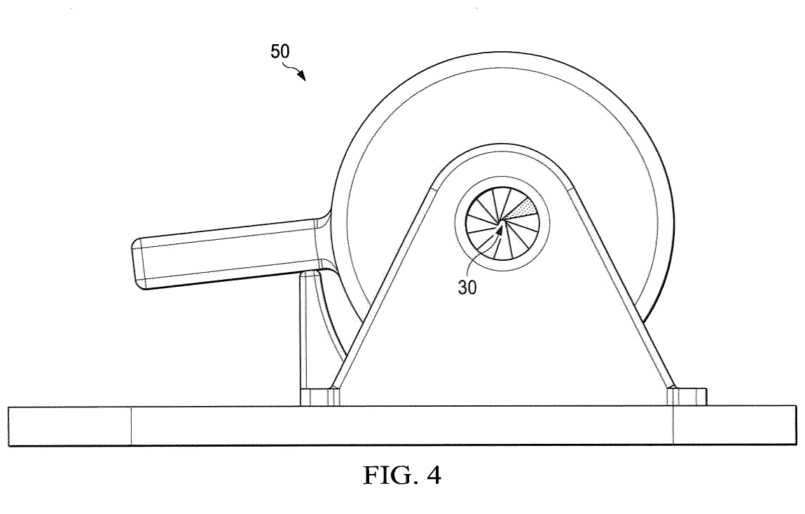

Referring now to FIGS. 3-4, a non-limiting crimper device 50 is illustrated that can be used to crimp the prosthetic valve. Crimper device 50 includes an opening 30 wherein the prosthetic valve is placed to be crimped.

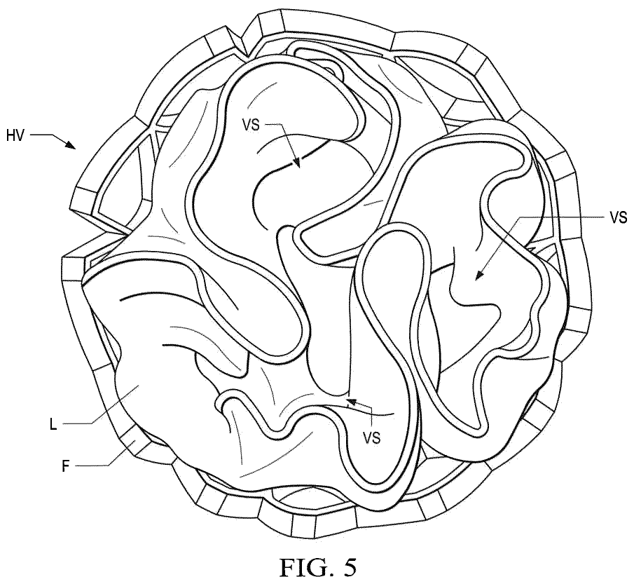

Referring now to FIG. 5, there is illustrated a rear-end view or outflow side end view of a crimped prosthetic valve that has been partially crimped by the crimper device and crimping process as disclosed herein. As illustrated in FIG. 5, the leaflets are illustrated as beginning to be folded in an organized manner that reduces the number and volume of void spaces existing about the folded leaflets.

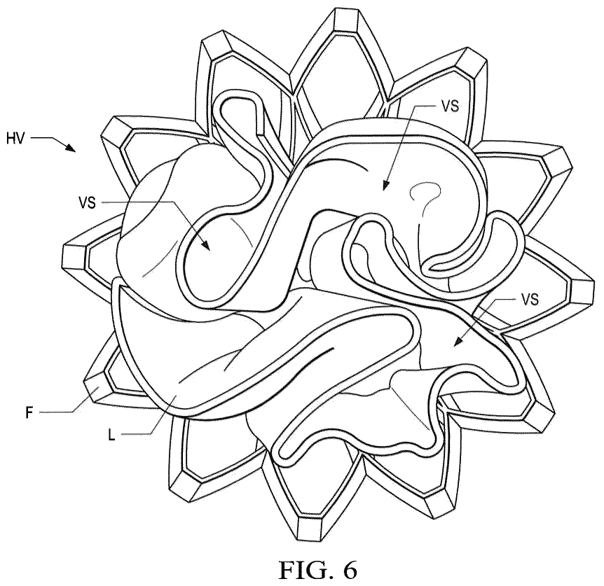

Referring now to FIG. 6, there is illustrated a rear-end view or outflow side end view of a crimped prosthetic valve showing the folding profile of leaflets in the fully crimped prosthetic valve that has been crimped by the crimper device and crimping process as disclosed herein. The prosthetic valve was crimped in such a way as to cause the leaflets to be folded in an organized manner that reduces the number and volume of void spaces about the folded leaflets, thereby enabling the frame of the prosthetic valve to be crimped to a smaller outer diameter.

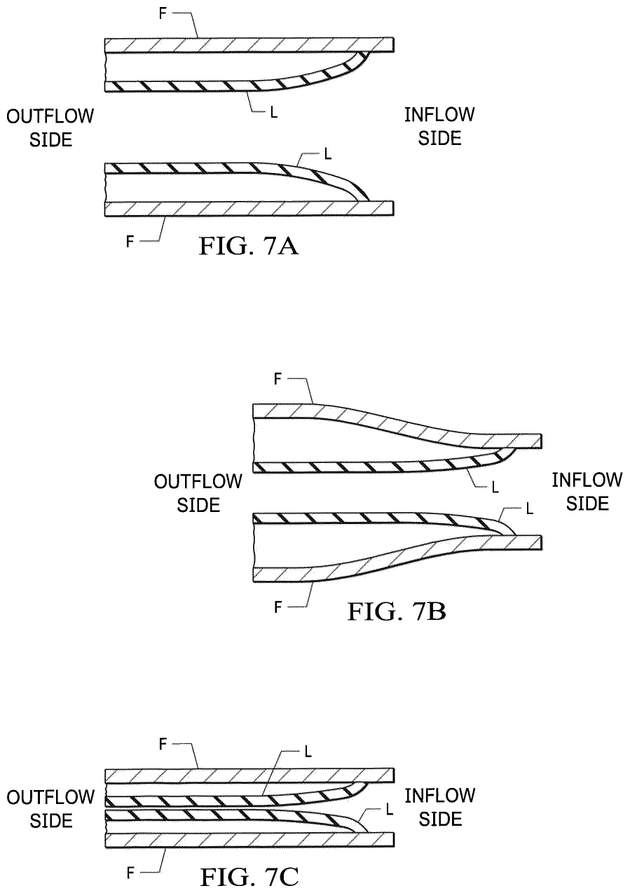

Referring now to FIGS. 7A-7C, there are illustrated three cross-sectional portions of a prosthetic valve along the longitudinal axis of the prosthetic valve wherein two leaflets are connected to the frame of the prosthetic valve. FIGS. 7A-7C illustrate the inflow side and outflow side of the prosthetic valve. The leaflets are illustrated as being connected to the frame at or near the inflow side of the prosthetic valve.

FIG. 7A illustrates a cross-sectional portion of the prosthetic valve prior to the frame being subjected to a crimping force by the crimper device. For example, when the handle is in the fully open position and the prosthetic valve is positioned in opening 30, the diameter or cross-sectional area of opening 30 can be such that no crimping force is applied to the prosthetic valve.

FIG. 7B illustrates a cross-sectional portion of the prosthetic valve wherein the inflow region of the frame has been subjected to a crimping force to cause a reduction in diameter or cross-sectional area of the crimped frame; however, the outflow region of the frame has not yet been subjected to a crimping force, thus there has been no reduction in diameter or cross-sectional area of this region of the frame. For example, as the handle is moved from the fully opened position to the fully closed or smallest diameter or cross-sectional area position for opening 30, the a first set of jaws of the crimper device can be configured to first contacting and applying a crimping force to the inflow region of the prosthetic valve prior to a second set of jaws contacting and applying a crimping force to the outflow region of the prosthetic valve.

FIG. 7C illustrates a cross-sectional portion of the prosthetic valve wherein both the inflow region and outflow regions of the frame have been subjected to a crimping force to cause a reduction in diameter or cross-sectional area of the crimped frame. For example, as the handle is continued to be moved to the fully closed or smallest diameter or cross-sectional area position for opening 30, the second set of jaws eventually contact and apply a crimping force to the outflow region of the prosthetic valve while the first set of jaws continue to apply a crimping force to the inflow region of the prosthetic valve.

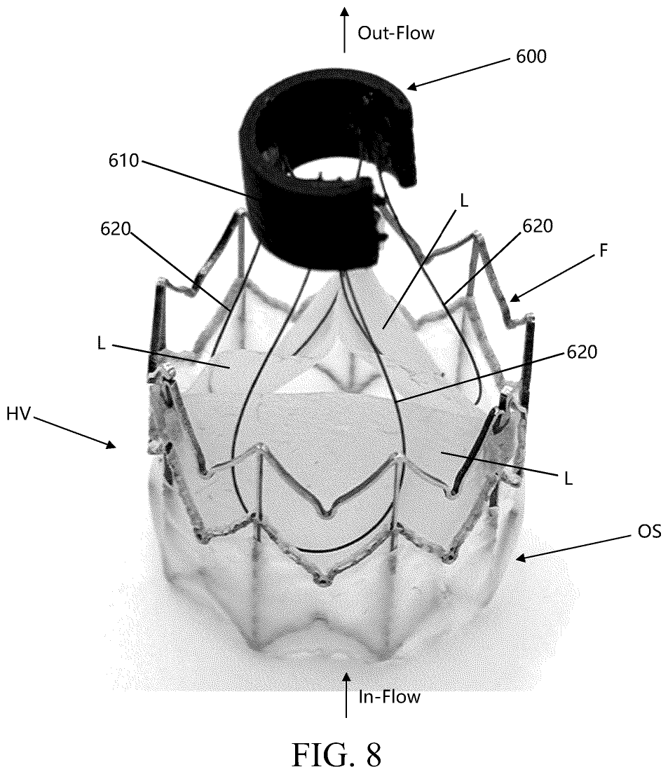

Referring now to FIG. 8, there is illustrated a prosthetic valve HV that includes a frame F, three leaflets L, and an inner skirt and an outer skirt OS. One non-limiting crimping accessory 600 is illustrated which can optionally be used with the crimper device to facilitate in a) obtaining a desired leaflet folding configuration or arrangement during the crimping of the frame of a prosthetic valve, and/or b) reduce or prevent damage to the leaflets L of the prosthetic valve HV during the crimping of the prosthetic valve HV. The crimping accessory 600 includes a handle 610 and three leaflet engagement members 620 in the form of wire loops (e.g., metal wire loop, plastic wire loop, etc.) that are attached to and extend from the handle 610. FIG. 8 only illustrates a portion of the handle 610. Generally, the handle 610 is sized and shaped to such that it can be grasped by a user to enable the user to position the leaflet engagement members into a portion of the prosthetic valve to cause the bending of the one or more leaflets. In one non-limiting embodiment, the handle is 2-20 inches in length (and all values and ranges therebetween). The shape of the handle is non-limiting along the longitudinal length of the handle (e.g., cylindrical, etc.). The material of the handle is also non-limiting (e.g., metal, plastic, wood, ceramic, etc.). The handle can be solid or hollow along a portion or all of the longitudinal length of the handle.

The leaflet engagement members 620 are attached to the distal end or distal end portion of the handle 610 and are illustrated as extending radially outwardly from the central longitudinal axis of the handle portion. In one non-limiting embodiment, the leaflet engagement members 620 extend about 10-50° (and all values and ranges therebetween) outwardly from the central longitudinal axis of the handle 610, and typically the leaflet engagement members 620 extend about 20-40° (and all values and ranges therebetween) outwardly from the central longitudinal axis of the handle 610. Generally, the leaflet engagement members 620 have the same size, shape, configuration, and/or can be formed of the same material; however, this is not required. Generally, the spacing of adjacently positioned leaflet engagement members 620 from one another is the same. As illustrated in FIG. 8, each of the leaflet engagement members 620 is formed of a looped member that forms a generally balloon-shape; however, other shapes can be formed (e.g., circular-shape, oval shape, etc.). The solid portion of the looped member generally constitutes 1-20% (and all values and ranges therebetween) of the total cross-sectional area of the looped member; however, this is not required. The longitudinal length of each of the leaflet engagement members 620 is generally less than the longitudinal length of the handle 610. The longitudinal length of each of the leaflet engagement members 620 is generally 20-150% (and all values and ranges therebetween) the longitudinal length of the frame 110 of the prosthetic valve HV. Generally, the leaflet engagement members 620 are flexible relative to the handle 610. The maximum width of the each of the leaflet engagement members 620 is generally 20-120% (and all values and ranges therebetween) the maximum width of a leaflet when the frame 110 of the prosthetic valve HV is in the expanded orientation prior to being crimped.

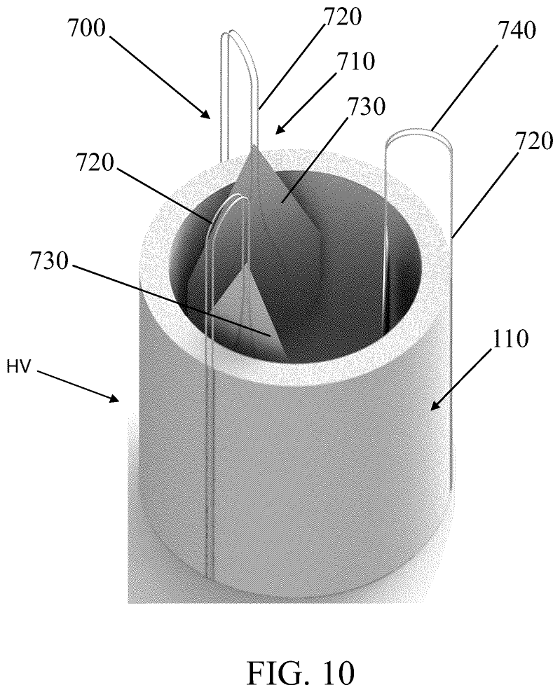

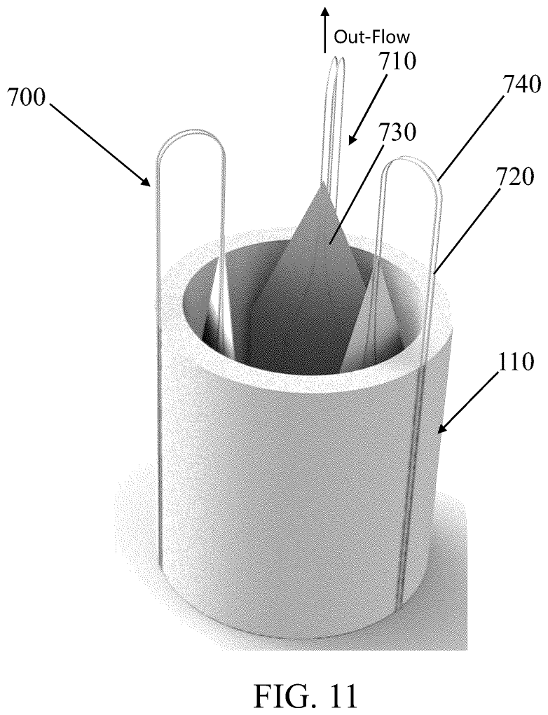

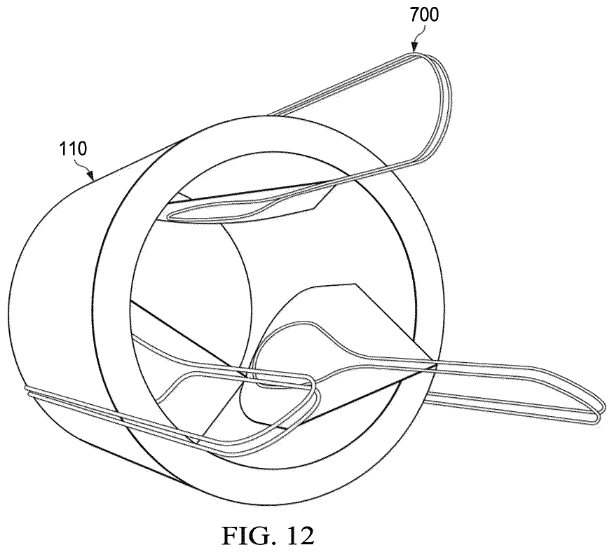

As will be discussed in more detail with respect to the leaflet engagement member illustrated in FIGS. 10-12, it will be appreciated that a portion or all of the looped member of the leaflet engagement member 620 can be modified to include the panel portions 730 illustrated in FIGS. 10-12. In such a non-limiting embodiment, the looped members can optionally provide sufficient strength and rigidity to the panel portion 730. As can be appreciated, the panel portions 730 on the looped members can be adjusted so that the panel portions 730 on the looped members are positioned between the leaflets and the frame prior to and/or during the crimping of the frame. The manner in which the panel portions are connected to the looped members is non-limiting (e.g., adhesive, melted connection, etc.).

The leaflet engagement members 620 can be formed of a flexible material that enables the leaflet engagement members to flex and/or bend a) when positioning the leaflet engagement members about one or more leaflets, and/or b) during the crimping of the frame of the prosthetic valve and while the leaflet engagement members are still engaged with the one or more leaflets during the crimping of the frame. In one non-limiting embodiment, the leaflet engagement members 620 can be formed of a flexible material such that the width of at least a portion of the leaflet engagement members 620 is reduced in size (e.g., 5-90% width reduction and all values and ranges therebetween) during the crimping of the frame; however, this is not required. In another non-limiting embodiment, the leaflet engagement members 620 are flexible relative to the handle such angle to when the leaflet engagement members 620 extends outwardly from the central longitudinal axis of the handle 610 is reduced (e.g., 5-95% angle reduction and all values and ranges therebetween) during the crimping of the frame; however, this is not required.

The crimping accessory is configured to inwardly bend one or more or all of the end portions of the leaflets that are located at or near the outflow end of the prosthetic valve toward the central axis of the frame of the prosthetic valve. Such bending of the one or more leaflets by the crimping accessory generally occurs a) prior to the initial crimping of the frame of the prosthetic valve, and/or b) during the crimping of the frame of the prosthetic valve. Generally, the crimping accessory is removed from or disengaged from the one or more leaflets prior to the outflow end of the frame of the prosthetic valve being fully crimped to as to not interfere with the complete crimping of the frame of the prosthetic valve.

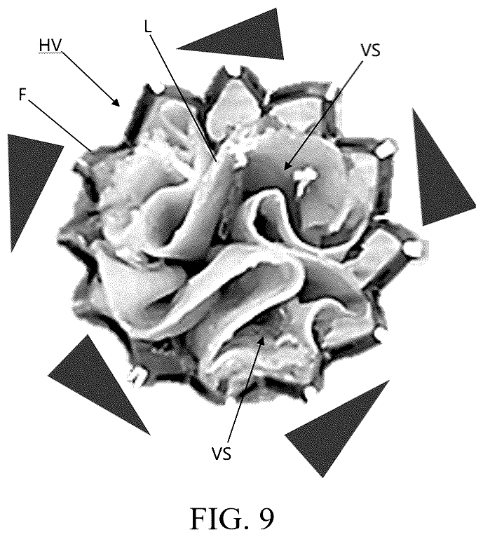

When the crimping accessory 600 is optionally used, one non-limiting method of use is as follows: a) the crimping accessory is moved along the longitudinal axis of the frame and toward the frame until the one or more leaflet engagement members engage the end or end portion of the one or more leaflets, b) thereafter, the crimping accessory is continued to be moved along the longitudinal axis of the frame such that i) the end or end portion of the one or more leaflet engagement members move between a portion or all of the one or more leaflets and the inner surface of the frame, and/or ii) the angular orientation of the one or more leaflet engagement members relative to the central axis of the handle portion of the crimping accessory causes the end and end portions of the leaflets to be bent toward the central axis of the frame. Generally, one or more leaflet engagement members are inserted only through a portion of the longitudinal length of the frame and are spaced from the region of the frame wherein the one or more leaflets are connected to the frame. Prior to and/or during the crimping of the frame, the handle portion of the crimping accessory can be optionally rotated about the longitudinal axis of the frame so as to facilitate in the folding of the leaflets during the crimping of the frame. Such rotation is illustrated by the arrows in FIG. 9. As illustrated in FIG. 9, the folding of the leaflets L is organized, and there are no significant void spaces VS about the folded leaflets L in the crimped prosthetic valve HV.

After the partial or full crimping of the frame, the crimping accessory is removed from the frame. In one non-limiting embodiment, the crimping accessory is removed from the frame prior to the full crimping of the frame.

Referring now to FIGS. 10-12, there is illustrated another non-limiting crimping accessory 700. The crimping accessory 700 includes typically includes a handle portion, not shown, and a plurality of leaflet engagement members 710. Generally, the number of leaflet engagement members 710 is generally the same number of leaflets in the prosthetic valve HV. As can be understood by one stilled in the art, the handle of crimping accessory 700 can be the same or similar to the handle described above with respect to FIG. 8.

As illustrated in FIGS. 10-12, each leaflet engagement member 710 includes two flexible wire portions 720 and a panel portion 730 connected to an end portion of the two flexible wire portions 720. As can be appreciated, the flexible wire portion 720 can alternatively formed of a wire loop, a single wire, three of more wires, a tubular member, etc. The flexible wire portion 720 can be formed of any type of flexible material (e.g., metal, plastic, composite material, etc.). The shape and size of the flexible wire portion 720 is non-limiting.

The panel portion 730 is illustrated as being positioned on the end or end portion of the flexible wire portion 720. The shape and size of the panel portion 730 is non-limiting. The panel portions 730 are generally formed of a flexible material (e.g., foam, plastic, metal, composite material, ceramic material, bovine pericardial tissue, biocompatible synthetic materials, other suitable natural or synthetic materials, etc.).

As illustrated in FIGS. 10-12, the panel portion 730 is a planar member that is connected to a top surface of one or both sides of the end region of the flexible wire portion and/or partially or fully encapsulates the end region of the flexible wire portion. The panel portion 730 can be molded and/or extruded onto the end region of the flexible wire portion, be adhesively connected to the end region of the flexible wire portion, and/or connected by other means (e.g., mechanical connection, adhesive, melted connection, etc.). As illustrated in FIGS. 10-12, each of the panel portions 730 have generally the same shape and size; however, this is not required.

One non-limiting shape of the panel portion 730 illustrated in FIGS. 10-12 has a generally triangular top portion and a square or rectangular bottom portion such that the panel portion 730 narrows to a point at the top of the panel portion 730 and has a generally flat wide bottom edge at the base of the panel portion 730. The panel portion 730 is illustrated as encapsulating the end region of the flexible wire portion 720. The flexible wire portion 720 for each of the panel portion 730 is illustrated as being a loop wire wherein two wires are positioned closely together at the top region of the panel portion 730 and then the spacing of the wires in the wire loop portion optionally expands in the bottom region of the panel portion 730. Such increase in wire spacing provides structural support to the bottom region of the panel portion 730 so that the bottom region of the panel portion 730 has sufficient strength and rigidity to push and/or otherwise prevent the leaflets from pushing into the side openings of the frame when the frame is being crimped. As can be appreciated, the flexible wire portion 720 can have other shape and/or include other structures to provide sufficient strength and rigidity to the panel portion 730. The panel portion 730 can be formed of a variety of materials (e.g., foam, plastic, metal, composite material, ceramic material, bovine pericardial tissue, biocompatible synthetic materials, other suitable natural or synthetic materials, etc.). In one non-limiting configuration, the panel portion 730 is formed of a polymer foam material or plastic material. The panel portion 730 can be formed of a material and/or coated with a material that has a low COF (e.g., coefficient of friction (COF) of 0.01 to 0.6 and all values and ranges therebetween; COF of 0.02-0.2) so that the panel portion 730 does not damage the leaflets during the crimping of the frame and/or adversely interfere with the folding of the leaflets during the crimping of the frame. The panel portion 730 and/or the flexible wire portion 720 can optionally include a marker (e.g., color region, structural marker, etc.) that can be used to facilitate in the positioning of the panel portions 730 relative to the leaflets of the medical device prior to and/or during the crimping of the frame of the medical device. The color of the panel portions 730 is non-limiting (e.g., clear, translucent, opaque, colored, patterned, etc.). As illustrated in FIGS. 10-12, the spacing between adjacently positioned panel portions 730 is generally the same; however, this is not required. In another non-limiting embodiment, the maximum width of the each of the panels on the leaflet engagement members is generally 20-120% (and all values and ranges therebetween) the maximum width of a leaflet when the frame 110 of the prosthetic valve HV is in the expanded orientation prior to being crimped. In another non-limiting embodiment, each of the panels on the leaflet engagement members is formed of a flexible material such that at least a portion of the panel is caused to bend during the crimping of the frame.

As illustrated in FIG. 10-12, the flexible wire portion 720 that extends from the panel portions 730 has a U-shaped region 740 that allows the flexible wire portion 720 to curve about the end of the frame 110 of medical device. Such non-limiting configuration of the flexible wire portions 720 is used when the outflow end of the medical device faces inwardly into opening 30 in the crimper device 50. When the outflow end of the medical device faces outwardly from opening 30 in the crimper device 50, the U-shaped region 740 of the flexible wire portion 720 is not required.

As illustrated in FIG. 10, the leg of the flexible wire portion 720 that includes the panel portion 730 is shorter than the other leg of the flexible wire portion 720. In one non-limiting embodiment, the leg of the flexible wire portion 720 that includes the panel portion 730 is 1.5 to 50 times shorter (and all values and ranges therebetween) than the other leg of the flexible wire portion 720.

As illustrated in FIG. 10, 5-100% (and all values and ranges therebetween) of the longitudinal length of the flexible wire portion 720 is formed of two wires that are positioned generally parallel to one another. As can be appreciated, the flexible wire portion 720 can be formed of a single wire or more than two wires.

In one non-limiting embodiment, the longitudinal length of each of the panel portions is generally 20-150% (and all values and ranges therebetween) the longitudinal length of the frame 110 of the prosthetic valve HV.

As illustrated in FIG. 10, the longitudinal axis of each of the panel portions is positioned generally parallel to the central longitudinal axis of the frame prior to the crimping of the frame; however, this is not required. In another non-limiting embodiment, the panel portions are configured to bend as the frame is crimped during the crimping process, and the panel portions optionally remain generally parallel to the central longitudinal axis of the frame during the crimping of the frame; however, this is not required.

In one non-limiting operation, the crimper device 50 and prosthetic valve HV are configured such that the prosthetic valve HV is to be positioned in the crimper device 50 such that the outflow end of the prosthetic valve HV faces inwardly into opening 30 in the crimper device 50 during the crimping process. For such a configuration, a user grasps the handle of the crimping accessory 700 (not shown) and positions the panel portions 730 on the end regions of the flexible wire portion 720 between the leaflets and the frame a) prior to the prosthetic valve HV being positioned in the opening 30 in the crimper device 50, b) after the prosthetic valve HV has been positioned in the opening 30 in the crimper device 50, c) prior to the crimping of the frame, and/or d) during the crimping of the frame. The crimping accessory 700 includes the U-shaped legs as illustrated in FIGS. 10-12 so that the handle portion of the crimping accessory 700 can be located rearwardly of the inflow end of the prosthetic valve HV and spaced from the opening 30 in the crimper device when the prosthetic valve HV is positioned in the opening 30 in the crimper device. After the partial or full crimping of the frame, the user can cause the panel portions 730 to be removed from the prosthetic valve HV. Generally, the removal of the panel portions 730 from the prosthetic valve HV first requires the prosthetic valve HV to be removed from the crimping device. Once the panel portions 730 are removed from the prosthetic valve HV, the prosthetic valve HV can optionally be reinserted into the crimping device for further crimping of the prosthetic valve HV.

In another non-limiting operation, the crimper device 50 and prosthetic valve HV are configured such that the prosthetic valve HV is to be positioned in the crimper device 50 such that the outflow end of the prosthetic valve HV faces outwardly in opening 30 in the crimper device 50 during the crimping process. For such a configuration, a user grasps the handle of the crimping accessory 700 (not shown) and positions the panel portions 730 on the end regions of the flexible wire portion 720 between the leaflets and the frame a) prior to the prosthetic valve HV being positioned in the opening 30 in the crimper device 50, b) after the prosthetic valve HV has been positioned in the opening 30 in the crimper device 50, c) prior to the crimping of the frame, and/or d) during the crimping of the frame. The crimping accessory 700 does not have the U-shaped legs as illustrated in FIGS. 10-12 since such U-shaped legs are not required to enable the handle portion of the crimping accessory 700 to be located rearwardly of the outflow end of the prosthetic valve HV and spaced from the opening 30 in the crimper device when the prosthetic valve HV is positioned in the opening 30 in the crimper device. After the partial or full crimping of the frame, the user can cause the panel portions 730 to be removed from the prosthetic valve HV. Generally, the removal of the panel portions 730 from the prosthetic valve HV does not require the prosthetic valve HV to be removed from the crimping device; however, the prosthetic valve HV can be removed when removing the panel portions. Once the panel portions 730 are removed from the prosthetic valve HV, the prosthetic valve HV can further crimped in the crimping device, or when the prosthetic valve HV was removed from the crimping device, the prosthetic valve HV is reinserted into the crimping device for further crimping of the prosthetic valve HV.

Frame 110 of the prosthetic valve HV is illustrated in FIGS. 10-12 as a hollow cylinder; however, such representation is merely used as a reference of how the crimping accessory 700 interacts with the frame 110. It will be understood by one skilled in the art that frame 110 illustrated in FIGS. 10-12 can have a shape and configuration that is the same or similar to the frame illustrated in FIGS. 1-2, 5-6 and 8, and/or as described above. Likewise, it will be understood by one skilled in the art that frame 110 as illustrated in FIGS. 10-12 includes one or more leaflets as illustrated in FIGS. 1-2, 5-6 and 8, and/or as described above, and that the panel portions 730 are configured to be positioned between at least a portion of one or more leaflets and a portion of the inner surface of the frame.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, Applicant does not intend any of the appended claims or claim elements to invoke 35 U.S.C. 112 (f) unless the words “means for” or “step for” are explicitly used in the particular claim.

What is claimed:

1. A method for reducing the outer diameter of a crimped prosthetic valve comprising:
providing a prosthetic valve; said prosthetic valve includes a frame, a leaflet structure supported by the frame; said leaflet structures include a plurality of leaflets; said frame in a non-fully crimped state;
providing a diameter reducing device that is configured to reduce an outer diameter of said prosthetic valve;
at least partially inserting said prosthetic valve in said diameter reducing device;
providing a crimping accessory; at least a portion of said crimping accessory is configured to be inserted between a portion of one or more of said plurality of leaflets and said frame;
at least partially inserting said crimping accessory inside at least a portion of said prosthetic valve prior to or during said step of initially reducing such that at least a portion of said crimping accessory is portioned between said one or more of said plurality of leaflets and said crimpable or plastically deformable portion of said medical device to facilitate in a) bending at least a portion of one or more of said leaflets toward said central longitudinal axis of said crimpable or plastically deformable portion, b) inhibiting or preventing at least a portion of one or more of said leaflets from being positioned in a side opening of said frame; and/or c) rotating at least a portion of one or more of said leaflets about said central longitudinal axis of said crimpable or plastically deformable portion;
reducing a diameter or cross-sectional area of at least a portion of said frame while said crimping accessory is at least partially inserted in said prosthetic valve; and
removing said crimping accessory from said prosthetic valve prior to completion of step of initially reducing.

2. The method as defined in claim 1, wherein said method further includes:
initially reducing an outer diameter of said prosthetic valve by use of said diameter reducing device; and
wherein said step of initially reducing includes applying a crimping force on said frame of the prosthetic valve along the longitudinal length of the prosthetic valve; and
wherein said step of initially reducing includes A) gradual application of said crimping force in a continuously progressive manner along a longitudinal length of said frame by i) initially applying said crimping force at an inflow end of said frame and subsequently applying said crimping force at locations that are spaced from said inflow end of said frame until said crimping force is applied to a complete outer surface of said frame along said longitudinal length of said frame; or ii) initially applying said crimping force at an outflow end portion of said frame and subsequently applying said crimping force at locations that are spaced from said outflow end portion of said flame until said crimping force is applied to a complete outer surface of said frame along said longitudinal length of said frame; or B) stepwise application of said crimping force along a longitudinal length of said frame by i) initially applying said crimping force at an inflow region of said frame until a portion or all of said inflow region of said frame is crimped to 10-100% of a fully crimped diameter or cross-sectional area, and then subsequently applying said crimping force at one or more locations that are spaced from said inflow region of said frame to crimped said one or more locations that are spaced from said inflow region of said frame to 10-100% of a fully crimped diameter or cross-sectional area, and wherein said inflow region of said frame extends from an inflow end of said frame to 5-75% of a longitudinal length of said frame; or ii) initially applying said crimping force at an outflow side of said frame until a portion or all of a outflow region of said frame is crimped to 10-100% of a fully crimped diameter or cross-sectional area, and then subsequently applying said crimping force at one or more locations that are spaced from said outflow region of said frame to crimped said one or more locations that are spaced from said outflow region of said frame to 10-100% of a fully crimped diameter or cross-sectional area, and wherein said outflow region of said frame extends from an outflow end of said frame to 5-75% of a longitudinal length of said frame.

3. The method as defined in claim 1, wherein said prosthetic valve is subjected to a further secondary crimping process after said step of initially reducing; said secondary crimping process includes a) simultaneously subjecting said prosthetic valve to said crimping force along 80-100% of said longitudinal length of said frame, b) said gradual application of said crimping force in a continuously progressive manner along 80-100% of said longitudinal length of said frame, or c) said stepwise application of said crimping force along 80-100% of said longitudinal length of said frame.

4. The method as defined in claim 1, further including the step of a) moving said crimping accessory along said longitudinal length of said frame during said step of initially reducing while said crimping accessory is at least partially inserted in said prosthetic valve, and/or b) rotating said crimping accessory in said frame during said step of initially reducing while said crimping accessory is at least partially inserted in said prosthetic valve.

5. The method as defined in claim 1, wherein said crimping accessory includes a handle portion and one or more leaflet engagement members that are attached to and extend from said handle portion; said one or more leaflet engagement members are at least partially formed of a flexible material that enables said one or more leaflet engagement members to flex and/or bend a) when positioning said one or more leaflet engagement members adjacent to one or more of said leaflets, and/or b) as said crimpable or plastically deformable portion of said medical device reduces in diameter or cross-sectional area as said crimping force is applied on said crimpable or plastically deformable portion of said medical device.

6. The method as defined in claim 5, wherein each of said leaflet engagement members includes a flexible wire portion and a panel portion; each of said panel portions is configured to be positioned between at least a portion of at least one of said leaflets and said crimpable or plastically deformable portion of said medical device.

7. The method as defined in claim 5, wherein each of said leaflet engagement members includes one or more wires that extend both radially outward from said handle portion and extend forwardly from an end of said handle portion.

8. The method as defined in claim 5, wherein a number of said leaflet engagement members is equal to a number of said leaflets in said medical device.

9. The method as defined in claim 5, wherein said one or more leaflet engagement members include one or more wire loops.

10. The method as defined in claim 5, wherein a number of said leaflet engagement members is equal to a number of said leaflets in said medical device.

11. The method as defined in claim 6, wherein each of said panel portions is positioned on an end or an end portion said flexible wire portion.

12. The method as defined in claim 6, wherein each of said panel portions is a planar member that is a) connected to a top surface of an end region of said flexible wire portion and/or b) partially or fully encapsulates said end region of said flexible wire portion.

13. The method as defined in claim 6, wherein each of said flexible wire portions for each of said panel portion includes a loop wire wherein two wires are positioned closely together at a top region of said panel portion and then a spacing of said two wires in a wire loop portion expands in a bottom region of said panel portion so as to provide structural strength and/or rigidity to said bottom region of said panel portion.

14. The method as defined in claim 6, wherein each of said panel portions is formed of a) a flexible material, b) a material that has a low COF, and/or c) is coated with a material that has a low COF.

15. The method as defined in claim 6, wherein one or more of said flexible wire portions and/or said panel portions include a marker that can be used to facilitate in positioning of one or more of said panel portions relative to one or more of the leaflets and the frame.

16. The method as defined in claim 6, wherein each of said flexible wire portions has a U-shaped region that allows the flexible wire portion to curve about an end of the frame the prosthetic valve.

17. A crimping accessory that is configured to inhibit or prevent damage to one or more leaflets of a prosthetic valve during crimping of a frame of the prosthetic valve, and wherein at least a portion of each leaflet is connected to the frame of the prosthetic valve; said crimping accessory includes a handle portion, and a plurality of leaflet engagement members that are connected to or interconnected to the handle portion; each leaflet engagement member includes a flexible wire portion and a panel portion; each of said panel portions is configured to be positioned between at least a portion of at least one of said leaflets and the frame of the prosthetic valve.

18. The crimping accessory as defined in claim 17, wherein said flexible wire portion is formed of one or more of a wire loop, a single wire, three of more wires, and/or a tubular member.

19. The crimping accessory as defined in claim 17, wherein said flexible wire portion is formed of a metal, plastic, and/or composite material.

20. The crimping accessory as defined in claim 17, wherein each of said panel portions is positioned on an end or an end portion said flexible wire portion.

21. The crimping accessory as defined in claim 17, wherein each of said panel portions is a planar member that is a) connected to a top surface of an end region of said flexible wire portion and/or b) partially or fully encapsulates said end region of said flexible wire portion.

22. The crimping accessory as defined in claim 17, wherein each of said flexible wire portions for each of said panel portion includes a loop wire wherein two wires are positioned closely together at a top region of said panel portion and then a spacing of said two wires in a wire loop portion expands in a bottom region of said panel portion so as to provide structural strength and/or rigidity to said bottom region of said panel portion.

23. The crimping accessory as defined in claim 17, wherein each of said panel portions is formed of a) a flexible material, b) a material that has a low COF, and/or c) is coated with a material that has a low COF.

24. The crimping accessory as defined in claim 17, wherein one or more of said flexible wire portions and/or said panel portions include a marker that can be used to facilitate in positioning of one or more of said panel portions relative to one or more of the leaflets and the frame.

25. The crimping accessory as defined in claim 17, wherein one or more of said panel portions is clear, translucent, or opaque.

26. The crimping accessory as defined in claim 17, wherein a spacing between adjacently positioned panel portions is generally the same.

27. The crimping accessory as defined in claim 17, wherein each of said flexible wire portions has a U-shaped region that allows the flexible wire portion to curve about an end of the frame the prosthetic valve.
